(19) United States
(12) Patent Application Publication
(10) Pub. No.: US 2011/0118467 A1
(43) Pub. Date: May 19, 2011

(54) PROCESS FOR THE PREPARATION OF CLOPIDOGREL HYDROGEN SULFATE CRYSTALLINE FORM I

(75) Inventors: Giorgio Soriato, Caldiero(Verona) (IT); Roberto Brescello, Abano Terme (Padova) (IT); Daniele Urbani, Lendinara (Rovigo) (IT); Livius Cotarca, Cervignano del Friuli (Udine) (IT)

(73) Assignee: ZACH SYSTEM S.P.A., Bresso (Milano) (IT)

(21) Appl. No.: 12/996,990

(22) PCT Filed: Jun. 11, 2009
(86) PCT No.: PCT/EP2009/057228

§ 371 Date: Dec. 9, 2010

(30) Foreign Application Priority Data
Jun. 24, 2008 (EP) 08104529.6

(51) Int. Cl. C07D 495/04

(52) U.S. Cl. 546/114

(57) ABSTRACT

The present invention relates to a process for the preparation of clopidogrel and, more particularly, to an improved process for the preparation of clopidogrel hydrogen sulfate crystalline Form I by addition of dilute sulfuric acid to a solution of clopidogrel free base in butyl acetate.

The present invention relates to a process for the preparation of clopidogrel and, more particularly, to an improved process for the preparation of clopidogrel hydrogen sulfate crystalline Form I, by addition of dilute sulfuric acid to a solution of dextro-rotatory clopidogrel free base in butyl acetate.

The invention also discloses a process for the resolution of racemic clopidogrel free base into its active isomer carried out in the presence of a lower alkyl ester type solvent.

Clopidogrel hydrogen sulfate also known as clopidogrel bisulfate or methyl (+)-(S)-alpha-(2-chlorophenyl)-4,5,6,7-tetrahydrothieno[3,2-c]pyridine-5-acetate hydrogen sulfate of formula

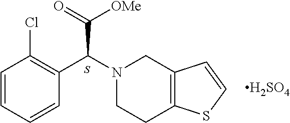

is an inhibitor of platelet aggregation. It is the active pharmaceutical ingredient contained in Sanofi-Aventis anticoagulant/thrombolytic drug PLAVIX®.

Clopidogrel hydrogen sulfate was first described in Sanofi U.S. Pat. No. 4,847,265 (U.S. '265) where it is stated that it has particular therapeutic properties compared to the known racemic form. The (+)—S-enantiomer has a better therapeutic index than the racemic mixture since the levo-rotatory isomer exhibits almost no platelet aggregation inhibiting activity and its toxicity is higher than that of the dextro-rotatory one. U.S. '265 discloses a method for the preparation of clopidogrel hydrogen sulfate, which comprises diastereoisomeric salt formation of racemic clopidogrel base with an optically active acid such as camphorsulfonic acid in acetone, followed by successive re-crystallization of the salt until a product with constant rotatory power is obtained. The salt is then transformed into free optically active base by reaction with a suitable base and converted to its hydrogen sulfate salt by reaction with concentrated sulfuric acid.

Polymorph forms of clopidogrel hydrogen sulfate are described in the subsequent Sanofi U.S. Pat. No. 6,429,210 (U.S. '210), which claims the stable crystalline Form II. In addition, U.S. '210 indicates that the process described in U.S. '265 for the preparation of clopidogrel hydrogen sulfate leads to a crystalline form designated as Form I.

Thus, in accordance with U.S. '265, crystalline Form I is prepared by dissolving dextro-rotatory clopidogrel base in acetone and adding sulfuric acid in equimolar amount to the solution at 20° C. The solvent is evaporated partly, the residue is cooled and the precipitate isolated by filtration.

Several alternative processes for the preparation of clopidogrel hydrogen sulfate Form I have been disclosed in the art.

WO 2003/051362 (Teva Pharm. Ind. LTD) discloses a process for obtaining crystalline Form I by contacting amorphous clopidogrel hydrogen sulfate with an ether, especially diethyl ether and methyl t-butyl ether.

WO 2004/048385 (Anpharm Przedsiebiorstwo Farmaceutyczne) describes a process for the synthesis of clopidogrel hydrogen sulfate, wherein crystalline Form I is prepared by reacting (S)-clopidogrel base with concentrated sulfuric acid followed by precipitating the salt from the media by the addition of a solvent selected from a group comprising aliphatic and cyclic ethers and isobutyl methyl ketone.

WO 2005/003139 (Egis Gyogyszergyar Rt.) discloses a method of preparation of clopidogrel hydrogen sulfate polymorph Form I which requires using two different solvents for the process of forming the bisulfate from clopidogrel base. The process comprises dissolving clopidogrel base in an “A” type solvent, preferably dichloromethane or acetone, adding sulfuric acid and adding the obtained mixture to a mixture of a “B” type solvent, preferably diisopropyl ether, cyclohexane or ethyl acetate, containing clopidogrel hydrogen sulfate polymorph Form I as a suspension. WO 2005/016931 (Krka, Torvara Zdravil, D. D., Novo Mesto) discloses a process for the preparation of Form I and Form II of clopidogrel hydrogen sulfate, a process for the preparation of amorphous clopidogrel hydrogen sulfate, the 2-propylsulfate of clopidogrel, the perchlorate of clopidogrel and methods for the preparation of these compounds.

WO 2005/063708 (Cadila Healthcare) describes a process for the preparation of Form I of clopidogrel bisulfate, which comprises treating clopidogrel base with either diluted or concentrated sulfuric acid in one or more suitable solvent(s) selected from C₆-C₁₂ alcohols with or without water.

WO 2005/104663 (Ipca Lab. LTD) discloses, inter alias, a process for resolution of racemic clopidogrel into its optical antipodes and the conversion of the dextro-clopidogrel base into its known polymorphs Form I or Form II in solvents selected from methyl propyl ketone, methyl isopropyl ketone, diethyl ketone or their mixtures, mixtures of ethyl acetate and methyl propyl ketone, mixtures of ethyl acetate and methyl isopropyl ketone or mixtures of ethyl acetate and diethyl ketone or ethyl acetate. In particular, Form I without detectable contamination of Form II is prepared by dissolving (+)-clopidogrel base in ethyl acetate at room temperature, cooling to 18° C. and adding concentrated sulfuric acid, provided that the temperature is maintained in the range from 18° to 30° C.; whilst Form II without detectable contamination of Form I is prepared from the same solvent, ethyl acetate, at a temperature of from 45° C. to 50° C.

WO 2005/012300 (Wockhardt LTD) describes a process for the preparation of (+)-(S)-clopidogrel bisulfate Form I comprising contacting (+)-(S)-clopidogrel with a sulfuric acid solution in an ester solvent, particularly in an acetate solvent for a sufficient time to form (+)-(S)-clopidogrel bisulfate Form I and isolation of the product. The solvent of choice is ethyl acetate, sulfuric acid is added at room temperature and the contacting step is conducted at reflux temperature.

WO 2004/020443 (Leciva) describes a method for manufacturing clopidogrel hydrogen sulfate in crystalline Form I, wherein the compound is separated out of a solution of clopidogrel in the form of the free base or salt in a solvent selected from the series of primary, secondary or tertiary C₁-C₅ alcohols, their esters with C₁-C₄ carboxylic acids, or optionally of mixtures thereof. In particular, in Example 2 clopidogrel hydrogen sulfate is dissolved in butyl acetate at reflux temperature and crystalline Form I is precipitated out of the solution by cooling. In Examples 5 and 6 clopidogrel base is dissolved in butyl acetate and the so obtained solution is cooled down to 0-5° C. and inoculated with crystals of Form I; concentrated sulfuric acid is then added and the crystallized mixture is stirred at a temperature between 5 and 15° C., filtered and dried to give crystalline Form I.

The present inventors have found that the processes described in WO '443 suffer from some procedural drawbacks, which markedly compromise the industrial scale-up of the methods. In particular, they have observed that reproducing the salt formation by addition of concentrated sulfuric acid at low temperature according to the suggestion provided by WO '443, the obtained solid comes out as a gummy and sticky mass, which can not be conveniently stirred and only after raising the temperature of the reaction mixture to room temperature and prolonged stirring of said gummy mass, a mixture of amorphous form and crystalline forms comprising crystalline Form I can be recovered.

Nevertheless, according to WO '663 application, the process variant of dissolving clopidogrel hydrogen sulfate salt in solvents at high temperature and cooling to precipitate Form I resulted either in Form II or Form IV or their mixture with Form I. From the foregoing, it appears very difficult to achieve pure Form I substantially free from other crystalline and/or solid forms: the crystallization process is strongly influenced by the reaction conditions and media wherein it is carried out, so that even slightly variations of the same may give rise to unpredictable changes in the orientation of the provided crystalline forms.

In view of the above, it would be desirable to have an alternative reliable and reproducible process for the preparation of clopidogrel hydrogen sulfate Form I on industrial scale, which results in a defined crystalline structure in its pure state and endowed with a high enantiomeric purity.

The present inventors have now surprisingly found an improved process for the preparation of clopidogrel hydrogen sulfate, which overcomes the drawbacks of the prior art and allows obtaining the desired crystalline Form I in good yields.

It is therefore an object of the present invention a process for the preparation of clopidogrel hydrogen sulfate crystalline Form I characterized in that said crystalline form is precipitated out of a solution of methyl (+)-(S)-alpha-(2-chlorophenyl)-4,5,6,7-tetrahydrothieno[3,2-c]pyridine-5-acetate (clopidogrel base) in butyl acetate at a temperature comprised between 40-65° C. by the addition of dilute sulfuric acid.

The crystallization according to the invention takes place in the presence of an organic solvent namely butyl acetate, preferably, n-butyl acetate.

In one embodiment of the invention, clopidogrel hydrogen sulfate crystalline Form I is obtained by dissolving dextro-rotatory clopidogrel base in butyl acetate at room temperature, raising the temperature of the solution in the range comprised between 40-65° C. and precipitating the crystalline product therefrom by the addition of dilute sulfuric acid to the hot solution.

It is preferable to perform a slow/dropwise addition of dilute sulfuric acid to the butyl acetate solution while maintaining the temperature in the range of precipitation. In a preferred embodiment of the invention the addition of dilute sulfuric acid to the solution of dextro-rotatory clopidogrel base in butyl acetate is carried out for a time of about 1 hour.

Under the term “dilute sulfuric acid” is meant a sulphuric acid solution prepared by diluting concentrated sulphuric acid with butyl acetate until a concentration ranging from 10 to 15% by weight of sulfuric acid is reached in the solution.

Under the “slow/dropwise addition” definition, the industrial operation of addition in small portions is meant.

Under the term “room temperature” is meant a temperature ranging from 18 to 25° C.

Molar ratio of sulfuric acid according to the present invention is around 1.0 with regard to the dextro-rotatory clopidogrel base.

In a preferred embodiment of the invention, crystalline Form I is precipitated out of a hot (40-65° C.) solution of dextro-rotatory clopidogrel base in n-butyl acetate by slow adding dilute sulfuric acid.

Said dilute sulfuric acid is preferably prepared just before performing the last salification step, wherein clopidogrel hydrogen sulfate crystalline Form I is formed. Optionally, the precipitation of clopidogrel hydrogen sulfate crystalline Form I may be supported by seeding the solution containing the clopidogrel free base with small amounts of crystals of pure Form I, in order to facilitate the precipitation of the desired crystalline form.

Preferably, the solution is seeded at a temperature comprised between 40-65° C., before adding dilute sulfuric acid.

According to the invention, the temperature range of the precipitation reaction is comprised between 40 and 65° C.

Preferably, the reaction is carried out at a temperature comprised between 45 and 60° C.; more preferably, the reaction is carried out at a temperature ranging from 50 to 55° C.

Generally, the precipitation reaction is followed by a controlled cooling phase in order to assist the isolation of the product.

Clopidogrel hydrogen sulfate crystalline Form I precipitates during the addition of sulfuric acid and the so obtained suspension is stirred for some hours.

According to one embodiment of the invention once slow/dropwise addition of sulfuric acid is completed, the fluid suspension in butyl acetate is stirred for some hours, preferably 1-4 hours, while maintaining the temperature in the range of precipitation, i.e., a temperature comprised between 40-65° C.

Alternatively, the fluid suspension may be heated to a slightly higher temperature (around 90° C.) and maintained at such temperature for some hours.

The formed product is then isolated by conventional methods; for example, the suspension is cooled to room temperature, preferably to a temperature ranging from 18 to 22° C., filtrated, optionally washed with the precipitating solvent and finally dried.

It is another object of the present invention a process for the preparation of clopidogrel hydrogen sulfate Form I which comprises:
dissolving clopidogrel base in butyl acetate;
raising the obtained solution to a temperature comprised between 40-65° C.;
seeding with pure clopidogrel hydrogen sulfate crystalline Form I;
slow adding diluted sulfuric acid;
keeping the obtained fluid suspension at a temperature comprised between 40-65° C.;
cooling and filtering.

As told before, prior art processes to obtain crystalline Form I are not easily scaled up, e.g. to an industrial scale.

On the contrary, when the preparation of crystalline Form I is carried out according to the present invention, a pure crystalline solid is directly and efficiently formed giving a fluid suspension suitable for a conventional isolation of the end-product.

As a matter of fact, the selection of the crystallization solvent as well as, mainly, the temperature range of the invention allows obtaining optical and crystalline pure Form I, consistently reproducible in manufacturing plants on industrial scale.

Hence, the process object of the invention provides a simple and readily industrialized alternative preparation of crystalline pure clopidogrel hydrogen sulfate Form I starting from dextro-rotatory clopidogrel free base which is, in turn, easily obtained by conventional methods known in the art.

Clopidogrel base, i.e., methyl (+)-(S)-alpha-(2-chlorophenyl)-4,5,6,7-tetrahydrothieno[3,2-c]pyridine-5-acetate, may be prepared, for example, by resolution of the corresponding racemic clopidogrel base, i.e., methyl (±)-alpha-(2-chlorophenyl)-4,5,6,7-tetrahydrothieno[3,2-c]pyridine-5-acetate, according to the teachings disclosed in U.S. '265.

Racemic clopidogrel base may be prepared according to known methods such as, for example, by condensation of a thienopyridine derivative with an o-chlorophenyl acetate derivative in the presence of an organic solvent as disclosed in U.S. Pat. No. 4,529,596. In a second aspect of the invention, the process for the preparation of clopidogrel hydrogen sulfate Form I further comprises a selective crystallization of racemic clopidogrel base from R-(−)-10-camphorsulfonic acid in the presence of a lower alkyl ester type solvent, to give enantiomerically pure dextro-rotatory clopidogrel base.

As lower alkyl ester type solvent an acetate solvent such as, e.g., methyl acetate, ethyl acetate, propyl acetate, butyl acetate and the like is meant.

Clopidogrel base is prepared by reacting corresponding racemic compound with optically active camphorsulfonic acid in the presence of a lower alkyl ester type solvent, preferably, ethyl acetate.

If necessary, crude clopidogrel camphorsulfonate diastereoisomeric salt may be further purified with organic solvents according to known methods. Suitable solvents are halogenated solvents, ester solvents or mixture thereof.

In a preferred embodiment of the invention, said salt is purified with a dichloromethane:ethyl acetate mixture.

Diastereoisomeric camphorsulfonate salt, wherein chiral center has the desired optical configuration, is then neutralized to give said dextro-rotatory clopidogrel base according to conventional methods.

Generally, the free base is liberated by treating said salt with an aqueous solution of a weak base such as an alkaline carbonates or hydrogen carbonates in the presence of an organic solvent.

In a preferred embodiment of the invention, clopidogrel camphorsulfonate salt is dissolved in a mixture of water and butyl acetate. Aqueous sodium hydrogen carbonate is then added to give dextro-rotatory clopidogrel free base which is extracted and concentrated to residue.

The process of the present invention provides a resolution method very efficient from the industrial viewpoint. The desired enantiomer is easily isolated from the reaction mixture in good yields and high enantiomeric excess.

The use of a lower alkyl ester type solvent allows the resolution of racemic clopidogrel base in a single crystallization step.

In addition, the use of butyl acetate in both steps (neutralization and crystallization) enables to achieve a benefit by reducing time and costs of the process.

It is therefore readily apparent that the process of the present invention is advantageous with respect to those already described in the art.

A practical embodiment of the process object of the present invention comprises resolution of racemic clopidogrel base by selective crystallization with optically active camphorsulfonic acid in the presence of ethyl acetate to give correspondent diastereoisomeric salt; neutralization of said diastereoisomeric camphorsulfonate salt with aqueous sodium hydrogen carbonate in a water:butyl acetate mixture to give dextro-rotatory clopidogrel free base; dissolving said optically active base in butyl acetate and raising the obtained solution at a temperature comprised between 40-65° C.; seeding with pure clopidogrel hydrogen sulfate crystalline Form I; slow adding dilute sulfuric acid prepared by diluting concentrated sulphuric acid with butyl acetate until a concentration ranging from 10 to 15% by weight of sulfuric acid is reached; keeping the obtained fluid suspension at a temperature comprised between 40-65° C. for a few hours, preferably, 1-4 hours; cooling and filtering.

The invention is illustrated by reference to the accompanying drawings described below.

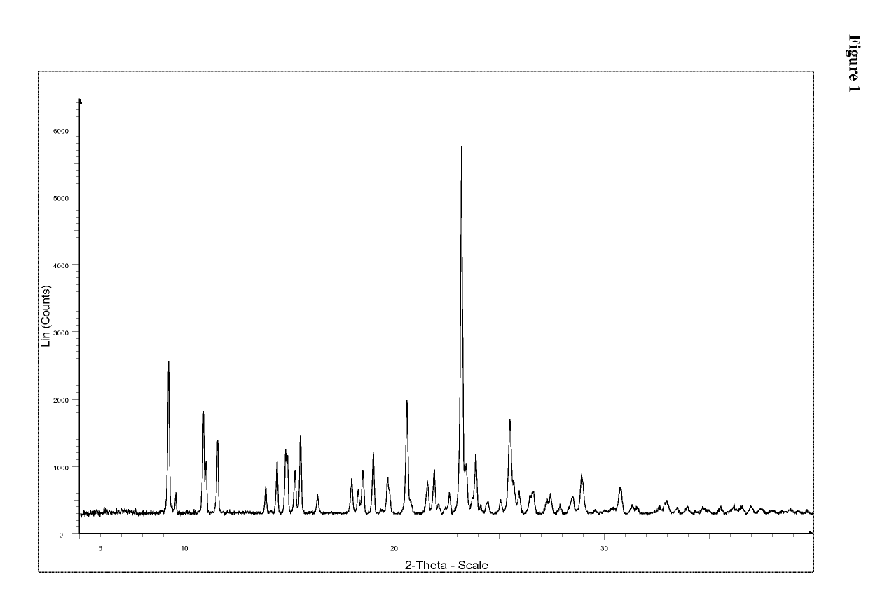

FIG. 1 depicts the X-ray powder diffractogram of clopidogrel hydrogen sulfate crystalline Form I prepared according to the process of the invention.

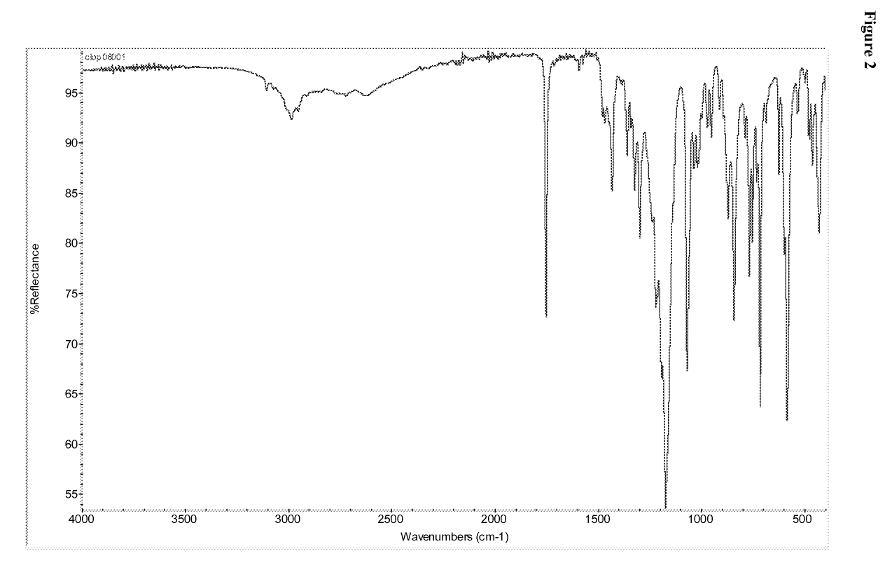

FIG. 2 depicts the IR spectrum of clopidogrel hydrogen sulfate crystalline Form I prepared according to the process of the invention.

The skilled person will realise that the relative intensity of the X-ray powder diffraction peaks can vary depending upon sample preparation technique, sample mounting procedure and the particular instrument employed.

It is to be understood that while the invention is described in conjunction of the preferred embodiments thereof, those skilled in the art are aware that other embodiments could be made without departing from the spirit of the invention.

For better illustrating the invention the following examples are now given.

### EXAMPLE 1

### Synthesis of (−)-(R)-camphorsulfonate salt of methyl (+)-(S)-alpha-(2-chlorophenyl)-4,5,6,7-tetrahydrothieno[3,2-c]pyridine-5-acetate

Methyl (±)-alpha-(2-chlorophenyl)-4,5,6,7-tetrahydrothieno[3,2-c]pyridine-5-acetate (100 g) and ethyl acetate (434 ml) were charged in a reactor. The obtained solution was heated to 70-75° C. and kept at that temperature till complete dissolution. (−)-(R)-camphorsulfonic acid (28.9 g) was charged by maintaining the temperature at about 70-75° C. The reaction mixture was seeded with a minimum amount of methyl (+)-(S)-alpha-(2-chlorophenyl)-4,5,6,7-tetrahydrothieno[3,2-c]pyridine-5-acetate (−)-(R)-camphorsulfonate salt maintaining the temperature at about 70-75° C. The obtained mass was aged at around 70-75° C. till precipitation completed and then cooled to around 20° C. After reaching that temperature, the mass was maintained at 20±1° C. for at least 2 h under stirring. The mass was then filtered and the cake was washed with ethyl acetate (51.0 ml) to give the title compound (105 g).

### EXAMPLE 2

### Synthesis of methyl (+)-(S)-alpha-(2-chlorophenyl)-4,5,6,7-tetrahydrothieno[3,2-c]pyridine-5-acetate

Methyl (+)-(S)-alpha-(2-chlorophenyl)-4,5,6,7-tetrahydrothieno[3,2-c]pyridine-5-acetate (−)-(R)-camphorsulfonate salt (1200 g), butyl acetate (1819.2 g) and demineralized water (909.6 g) were charged in a 20 l reactor and kept under stirring at room temperature (around 25° C.). Separately, a sodium hydrogen carbonate (219.6 g) and demineralized water (2733.6 g) solution was prepared and, then, added to the camphorsulfonate mixture. So obtained reaction mixture was maintained at room temperature for 1 h. Aqueous phase was separated and again extracted with butyl acetate (909.6 g). The collected organic phases were washed with demineralized water (909.6 g). So obtained organic phase was distilled under vacuum to give an oil residue which was diluted with butyl acetate (1020 g) and again distilled to give the title compound as an oil.

### EXAMPLE 3

### Synthesis of hydrogen sulfate salt of methyl (+)-(S)-alpha-(2-chlorophenyl)-4,5,6,7-tetrahydrothieno[3,2-c]pyridine-5-acetate crystalline Form I

Butyl acetate (5220 g) was added to the oil residue obtained in example 2 to give a solution which was cooled to 20° C. and filtered. The reactor, lines and filter were washed with further butyl acetate (960 g). Flashing solvent was collected with the filtered solution. The solution was heated to 50-55° C. and seeded with clopidogrel hydrogen sulfate Form I (44.4 g). To the so obtained suspension, dilute sulfuric acid, prepared by diluting concentrated (96%) sulfuric acid (218.4 g) with butyl acetate (1600 g), was charged in around 1 h by maintaining temperature at 50-55° C. At addition completed, lines were flashed with butyl acetate (180 g). The suspension was further maintained at 50-55° C. for 1 h and then cooled to 20° C. in about 1 h and filtered. The product was washed with butyl acetate (1248 g) to give a wet compound (1102 g) which was dried under vacuum (65-70° C.) to give the title clopidogrel hydrogen sulfate Form I (836.2 g).

1) A process for the preparation of clopidogrel hydrogen sulfate crystalline Form I characterized in that said crystalline form is precipitated out of a solution of methyl (+)-(S)-alpha-(2-chlorophenyl)-4,5,6,7-tetrahydrothieno[3,2-c]pyridine-5-acetate in butyl acetate at a temperature comprised between 40-65° C. by addition of dilute sulfuric acid.

2) A process according to claim 1, wherein Form I is precipitated out of a solution of methyl (+)-(S)-alpha-(2-chlorophenyl)-4,5,6,7-tetrahydrothieno[3,2-c]pyridine-5-acetate in n-butyl acetate.

3) A process according to claim 1, further comprising seeding with crystals of Form I.

4) A process according to claim 1, wherein the molar ratio of the sulfuric acid is 1.0 with regard to dextro-rotatory clopidogrel base.

5) A process according to claim 1, wherein Form I is precipitated out of a solution of methyl (+)-(S)-alpha-(2-chlorophenyl)-4,5,6,7-tetrahydrothieno[3,2-c]pyridine-5-acetate in butyl acetate at a temperature comprised between 45-60° C.

6) A process according to claim 5, wherein the temperature is comprised between 50-55° C.

7) A process according to claim 1 further comprising keeping Form I fluid suspension in butyl acetate at a temperature comprised between 40-65° C.

8) A process for the preparation of clopidogrel hydrogen sulfate Form I, which comprises:
dissolving clopidogrel base in butyl acetate;
raising the obtained solution to a temperature comprised between 40-65° C.;
seeding with pure clopidogrel hydrogen sulfate crystalline Form I;
slow adding dilute sulfuric acid,
keeping the obtained fluid suspension at a temperature comprised between 40-65° C.;
cooling and filtering.

9) A process according to claim 1, further comprising the resolution of racemic clopidogrel base by selective crystallization with R-(−)-10-camphorsulfonic acid in the presence of a lower alkyl ester type solvent.

10) A process according to claim 9, wherein said lower alkyl ester type solvent is ethyl acetate.
